(19) United States
(12) Patent Application Publication
(10) Pub. No.: US 2017/0035675 A1
(43) Pub. Date: Feb. 9, 2017

(54) USE OF ALKYL POLYGLYCOSIDES AS PERFUME-SOLUBILISING AGENTS AND PERFUME COMPOSITION INCLUDING SAME

(71) Applicant: SOCIETE D'EXPLOITATION DE PRODUITS POUR LES INDUSTRIES CHIMIQUES SEPPIC, Paris (FR)

(72) Inventors: Marie-Laure BRETON, Castres (FR); Sebastien KERVERDO, Vincennes (FR); Stephanie GARCEL, Castres (FR); Jerome GUILBOT, Castres (FR); Herve ROLLAND, Castres (FR)

(21) Appl. No.: 15/105,588

(22) PCT Filed: Dec. 3, 2014
(86) PCT No.: PCT/FR2014/053150

§ 371 Date: Jun. 17, 2016

(30) Foreign Application Priority Data
Dec. 18, 2013 (FR) 1362862

(51) Int. Cl. A61K 8/60; A61Q 13/00

(52) U.S. Cl. CPC A61K 8/604; A61Q 13/00; A61K 2800/49

(57) ABSTRACT

A composition represented by the formula (I): R-0-(G)ₚ-H, wherein R is the n-heptyl radical, G is the residue of a reducing sugar and p is a decimal number no lower than 1.05 and no higher than 5. The composition is a mixture of compounds represented by the formulas R-0-(G)₁-H (I₁), R-0-(G)₂-H (I₂), R—O-(G)₃-H (I₃), R—O-(G)₄-H (I₄), and R-0-(G)₅-H (I₅) in the respective molar ratios a₁, a₂, a₃, a₄ and a₅, such that each ratio a₁, a₂, a₃, a₄ and a₅ is between zero and one inclusive and that the sum of a₁+a₂+a₃+a₄+a₅, is equal to one. Also, the method of solubilizing, in an aqueous solution, a hydrophobic perfume substance (A) or a mixture of hydrophobic perfume substances (A) with this composition, and a solubilising composition for hydrophobic perfumes. Further, a method for perfuming surfaces.

The present invention relates to alkyl polyglycosides and to their use as solubilizing agents in an aqueous medium.

There is a recurring need in the cosmetics and perfumery industries for the development of perfuming compositions with reduced concentrations of volatile alcohols, or even for perfuming compositions which are free of volatile alcohols. Perfuming compositions of this type in fact suffer from a number of disadvantages linked to the presence of these volatile alcohols, and more particularly to the presence of ethanol, as they cause drying of the skin and can irritate the epidermis, they are sensitive to sunlight, and employing them in the field of fragrances is fraught with difficulty, since the odor of the volatile alcohol can interfere with the perfuming substances.

Water is a substitute of choice for volatile alcohols in perfuming compositions, because using it means that the quantity of volatile organic compounds in the atmosphere can be reduced, and the flammable nature of perfuming compositions can be reduced or eliminated. Furthermore, the perfumery and cosmetics industries also market compositions requiring the presence of water, such as eaux de toilettes, eaux de parfum, aqueous aftershave lotions body mists and colognes. These aqueous perfumed cosmetic products are appreciated by consumers as they are characterized by an increased sensation of freshness.

However, since perfuming substances are generally hydrophobic substances, it turns out to be necessary to use at least one third ingredient in order to solubilize them in the aqueous phase of the perfuming compositions. Surfactants are generally solubilizing agents which are associated with the hydrophobic perfuming substances in order to prepare aqueous perfuming compositions.

Included among surfactants which are in routine use are non-ionic alkoxylated surfactants such as polysorbates, ethoxylated esters of lauroyl sorbitan containing 20 moles of ethylene oxide, ethoxylated esters of palmitoyl sorbitan containing 20 moles of ethylene oxide, ethoxylated esters of stearoyl sorbitan containing 20 moles of ethylene oxide, ethoxylated esters of oleyl sorbitan containing 20 moles of ethylene oxide; ethoxylated fatty alcohols, ethoxylated fatty acids, ethoxylated fatty esters, and more particularly ethoxylated fatty triglycerides such as ethoxylated hydrogenated castor oil containing 20, 40 or 60 moles of ethylene oxide, and more particularly ethoxylated hydrogenated castor oil containing moles of ethylene oxide, which is currently commercially available under the commercial name “PEG-40 hydrogenated castor oil”. These ethoxylated surfactants, however, suffer from the disadvantage of being prepared using ethylene oxide; residual quantities of these and the by-products they generate are prohibited in cosmetic products for consumer use, which means that restrictive purification processes have to be carried out. In addition, it is suspected that polyethoxylated compounds can readily breach the skin barrier, and thus residual quantities of ethylene oxide and its by-products can more rapidly gain access to the human organism.

For these reasons, alternatives to alkoxylated solubilizing agents have been the focus of a great deal of research, involving structures with starting materials which are obtained from renewable resources. Examples of alternative structures to alkoxylated solubilizing agents that may be mentioned are N-acylated amino acid derivatives, polyglycerol esters, alkyl polyglycosides or mixtures of at least two of these compounds.

The international application published with the number WO 2010/106423 A2 discloses that the combination of polyglycerol esters of fatty acids and N-acyl glutamate derivatives can be used to obtain a solubilizing system which can effectively solubilize a wide range of lipophilic ingredients such as essential oils and perfumes in water.

The alkyl polyglycoside family includes caprylyl/caprylic glucoside, which is a compound which is routinely used to solubilize lipophilic chemical compositions and/or substances in water, and in particular to solubilize lipophilic perfuming substances in water. However, caprylyl/caprylic glucoside suffers from the disadvantage of generating the formation of foam in solution, and it has been shown to have limited capacity as regards providing aqueous perfuming compositions comprising it with transparency.

The solubilizing properties of alkyl polyglycosides may be improved by combining them with another surfactant such as a glycerol ester and/or an N-acylated derivative, and more particularly an N-acylated derivative, of a glutamic acid salt.

The international application published with the number WO 2010/106423 A1 discloses solubilizing compositions containing the combination of at least one polyglycerol ester of a fatty acid containing 8 or 10 carbon atoms and at least one N-acyl glutamate, in the salt form with an alkaline metal or with an alkaline-earth or with an ammonium ion or with an amine, and wherein the acyl radical contains 8 to 14 carbon atoms.

The international application published with the number WO 2010/106423 A1 also discloses the use of compositions of this type for solubilizing oils or perfumes in water. The disadvantage with this solution lies in the fact that the solubilizing composition only operates at pH values above 7.0, because the glutamic acid has to be solubilized completely in the N-acyl glutamate derivative.

The international application published with the number WO 96/14374 A1 discloses a surfactant system constituted by at least one alkyl polyglycoside wherein the alkyl chain contains 1 to 8 carbon atoms, and by at least one carboxylic acid N-alkylpolyhydroxyalkylamide, as well as the use of this surfactant system as a solubilizing agent in the preparation of cosmetic, pharmaceutical and detergent compositions. The international patent application published with the number WO 96/14374 A1 also discloses that the alkyl polyglycosides that are the constituents of the solubilizing surfactant system with a carboxylic acid N-alkylpolyhydroxyalkylamide are preferably the methyl oligoglycoside, the butyl oligoglycoside, the ethyleneglycol oligoglucoside and/or the glyceryl oligoglucoside.

The European patent application published with the number EP 2 366 376 A1 discloses a composition containing at least one alkyl polyglycoside the alkyl chain of which contains 4 to 12 carbon atoms, at least one polyglycerol ester of a fatty acid the alkyl chain of which contains 8 to 20 carbon atoms, at least one N-acylated derivative of an amino acid the acyl chain of which contains 6 to 12 carbon atoms, and at least one wetting agent the molecular weight of which is in the range 100 g·mol⁻¹ to 250 g·mol⁻¹, selected from the group constituted by monoesters of glycerol and fatty acids, the alkyl chain of which contains 6 to 10 carbon atoms, and 1,2-alkanediols the hydrocarbon chain of which contains 6 to 10 carbon atoms.

These various solutions are characterized by a finely tuned optimization of the proportions of the various constituents of the solubilizing compositions, placing the formulator of aqueous perfuming compositions in the difficult position of having to scale up complex mixtures and also of being faced with a multitude of commercial ingredients that constitute the solubilizing system, thereby generating supplemental operational costs.

The person skilled in the art is thus seeking a solution which uses just one substance or one solubilizing composition.

The French patent application published with the number FR 2 816 517 A1 discloses the preparation of alkyl polyglycosides starting from fusel alcohols and at least one reducing sugar in an acidic catalytic medium, and the use of said alkyl polyglycoside on fusel alcohol as a solubilizing agent for oily or liposoluble ingredients. Fusel alcohols are mixtures of alcohols primarily comprising short chain alcohols such as ethanol, propan-1-ol, propan-2-ol, methyl-propan-1-ol, butan-1-ol, 2-methyl butan-1-ol and 3-methyl butan-1-ol, which necessitate the use of flameproof safety measures when used on an industrial scale. The French patent application published with the number FR 2 816 517 A1 also discloses that alkyl polyglycosides, the alkyl chain of which originates from fusel alcohol, can be used to “increase the solubility of lipophilic molecules in water” such as essential oils and perfumes. However, alkyl polyglycosides prepared from fusel alcohols are characterized by an odor which means that they are not very compatible with formulations for compositions comprising perfuming substances.

The international application published with the number WO 96/33255 A1 describes anti-foaming compositions comprising a particular alkyl polyglycosides the alkyl chain of which is constituted by the 2-ethylhexyl radical, and non-ionic defoaming surfactants selected from those containing one or more groups selected from mono-ethoxylated or poly-ethoxylated groups, and mono-propoxylated or poly-propoxylated groups. It also discloses that alkyl polyglycosides with a 2-ethylhexyl chain are more effective than alkyl polyglycosides with an n-hexyl chain in solubilizing non-ionic defoaming surfactants.

The international application published with the number WO 99/21948 A1 discloses clear compositions that are stable at high alkaline concentrations, wherein the foaming properties are controlled, containing a large quantity of non-ionic surfactants based on alkylene oxide and a hexyl glycoside as a hydrotropic agent. It also discloses that hexyl glycosides, and more particularly n-hexyl polyglucoside, can be used to solubilize non-ionic surfactants in highly alkaline media.

The international application published with the number WO 2012/069730 A1 discloses clear compositions that are stable at high concentrations of both alkaline species and electrolytic species, comprising a large quantity of non-ionic surfactants based on alkylene oxide and n-heptyl polyglucoside as a hydrotropic agent.

Nothing in the prior art teaches that alkyl polyglycosides prepared from n-heptanol and known for their hydrotropic properties, namely of solubilizing surfactants which have poor solubility in highly alkaline or acid or electrolytic media in water, could constitute solubilizing agents for hydrophobic perfuming substances in water, not inducing the formation of a foam, which could be used in a pH range in the range 4 to 10, and which does not denature the odor provided by the solubilized perfuming substances.

For this reason, in a first aspect, the invention pertains to the use of a composition represented by the formula (I):

R—O-(G)ₚ-H (I)

in which R represents the n-heptyl radical, G represents the residue of a reducing sugar and p represents a decimal value greater than or equal to 1.05 and less than or equal to 5, said composition with formula (I) consisting of a mixture of compounds represented by the formulae (I₁), (I₂), (I₃), (I₄) and (I₅):

R—O-(G)₁-H (I₁)

R—O-(G)₂-H (I₂)

R—O-(G)₃-H (I₃)

R—O-(G)₄-H (I₄)

R—O-(G)₅-H (I₅)

in the respective molar proportions a₁, a₂, a₃, a₄ and a₅, such that each of the proportions a₁, a₂, a₃, a₄ and a₅ is greater than or equal to zero and less than or equal to one and such that the sum a₁+a₂+a₃+a₄+a₅ is equal to one, as a solubilizing agent in aqueous solution, for a hydrophobic perfuming substance (A) or for a mixture of hydrophobic perfuming substances (A), characterized in that said hydrophobic perfuming substance (A) is selected from the group constituted by essential oils that are rich in compounds carrying a phenolic function and essential oils that are rich in compounds carrying an alcohol function.

The term “reducing sugar” as used in formula (I) denotes saccharide derivatives the structure of which does not contain a glycoside bond between an anomeric carbon and the oxygen of an acetal group, as defined in the reference work: “Biochemistry”, Daniel Voet/Judith G. Voet, p. 250, John Wiley & Sons, 1990. The oligomeric structure (G)ₚ may be in any of the isomeric forms, irrespective of whether it is the optical isomer, geometrical isomer or positional isomer; it may also represent a mixture of isomers.

In formula (I) as defined above, the R—O group is bonded to G via the anomeric carbon of the saccharide residue, in a manner such as to form an acetal function.

The term “hydrophobic perfuming substance (A)”, when used in the manner defined above, means any hydrophobic perfume that is capable of releasing an odor of greater or lesser persistency, said perfume belonging to one of the fragrance families as established by the French Perfumer's Society. This classification is known to the person skilled in the art and is, for example, published on the website of this society, at the following address: http://www.parfumeurs-createurs.org/gene/main.php?base=525

This classification consists of a list of the following fragrance families: fresh family, floral family, fougere family, chypre family, woody family, floriental family and leather family.

Associating various hydrophobic perfuming substances means that a pleasant olfactive signature can be produced which persists to a greater or lesser extent.

The hydrophobic perfuming substance (A) may be selected from hydrophobic perfuming substances of natural, animal, or plant origin, or of synthetic origin.

Hydrophobic perfuming substances of animal origin include musk, castoreum, civet, ambergris, beeswax absolute, and hyraceum.

The hydrophobic perfuming substances of plant origin may be extracted from various parts of plants such as the petals, leaf, stem, bark, wood, wood moss, fruit, bud, seed, root, grass, plant resins or lichens.

Hydrophobic perfuming substances of plant origin obtained by extraction from petals include those obtained by extraction from rose petals, jasmine petals, tuberose petals, champaca petals, ylang-ylang petals, lotus petals, mimosa petals, carnation petals, osmanthus petals, acacia petals, orange petals, narcissus petals, lavender petals, and gardenia petals.

Hydrophobic perfuming substances of plant origin obtained by extraction from buds include those obtained by extraction from blackcurrant buds.

Hydrophobic perfuming substances of plant origin obtained by extraction from mosses include those obtained by extraction from oak moss or from beech moss.

Hydrophobic perfuming substances of plant origin obtained by extraction from leaves include hydrophobic perfuming substances obtained by extraction from acacia leaves, basil leaves, valerian leaves, gentian leaves, violet leaves, geranium leaves, labdanum leaves, rosemary leaves, patchouli leaves and verbena leaves.

Hydrophobic perfuming substances of plant origin obtained by extraction from bark include hydrophobic perfuming substances obtained by extraction from cinnamon bark, ash bark, cassia bark, cascarilla bark, and birch bark.

Hydrophobic perfuming substances of plant origin obtained by extraction from wood include hydrophobic perfuming substances obtained by extraction from sandalwood, cedar wood, rose wood, agar wood, and gaiac wood.

Hydrophobic perfuming substances of plant origin obtained by extraction from resins include hydrophobic perfuming substances obtained by extraction from labdanum resin, elemi resin, Peru balsam, Tolu balsam, benzoin resin, and myrrh tree resin.

Hydrophobic perfuming substances of plant origin obtained by extraction from resins include incense, opoponax, and guggul.

Hydrophobic perfuming substances of plant origin obtained by extraction from herbs or grasses include hydrophobic perfuming substances obtained by extraction from tarragon, sage, thyme, basil and lemon grass.

Hydrophobic perfuming substances of plant origin obtained by extraction from roots include hydrophobic perfuming substances obtained by extraction from angelica, celery, cardamom, iris, sweet flag, cactus, and vetiver roots.

Hydrophobic perfuming substances of plant origin obtained by extraction from fruits or seeds include hydrophobic perfuming substances obtained by extraction from vanilla pods, coriander seeds, star anise, fennel seeds, juniper berries, cardamom pods, cumin seeds, cloves, tonka beans, bitter almonds, citrus fruit such as lemon, lime, orange or mandarin, and bergamot.

The hydrophobic perfuming substances of plant origin include extracts, absolutes, alcoholates and essential oils.

The term “essential oil” as used in the present application means a fragrancing product complying with the definition in ISO standard 9235, generally with a complex composition, obtained from a botanically defined plant starting material either by entrainment in steam, or by dry distillation, or by an appropriate mechanical process without heating. The essential oil is generally separated from the aqueous phase by a physical process which does not involve a significant change in its composition. The plant starting material employed to obtain the essential oil may be fresh, wilted, dry, whole, bruised or pulverized. The essential oil may also undergo an appropriate subsequent process step such as a subsequent process step which can produce a deterpenated, desesquiterpenated, rectified essential oil, or an essential oil that is depleted in a named and identified constituent, or a decolorized essential oil.

The term “deterpenated essential oil” means an essential oil as defined above which is partially or completely free of monoterpene hydrocarbons.

The term “desesquiterpenated essential oil” means an essential oil as defined above which has been partially or completely freed from monoterpene and sesquiterpene hydrocarbons.

The term “rectified essential oil” means an essential oil as defined above which has undergone a fractional distillation with the aim of removing certain constituents or of modifying the content thereof.

The essential oils as defined above comprise a mixture of different molecules and said essential oils may be classified into different families depending on the chemical nature of their major components. Thus, it is possible to define:
the class of essential oils that is rich in terpene carbides and in sesquiterpene carbides,
the class of essential oils that is rich in compounds carrying an alcohol or monoterpene alcohol function,
the class of essential oils that is rich in compounds carrying an ester function,
the class of essential oils that is rich in compounds carrying an aldehyde function,
the class of essential oils that is rich in compounds carrying a ketone function,
the class of essential oils that is rich in compounds carrying a phenolic function,
the class of essential oils that is rich in compounds carrying an ether function,
the class of essential oils that is rich in sesquiterpene oxides,
the class of essential oils that is rich in compounds carrying at least one sulfur atom.

The term “essential oils that are rich in compounds carrying an alcohol function” means essential oils comprising, with respect to 100% of their mass, a proportion by weight greater than or equal to 35% of compounds carrying an alcohol function.

Compounds carrying an alcohol function routinely present in essential oils include monoterpenols such as linalol, geraniol, menthol, neomenthol, sabinene cis-hydrate, citronnellol, α-terpineol, sesquiterpenols such as viridiflorol, trans-nerolidol, α-cadinol, Tcadinol, trans-farnesol, cis-farnesols, α-bisabolol, patchoulol or pogostol.

Essential oils that are rich in compounds carrying an alcohol function include bourbon geranium, Egyptian geranium, lavender aspic, rosalina, coriander, rose wood, rose petals, palmarosa, peppermint, monarda fistulosa, lavandin super, lavandin grosso, or cabreuva.

The term “essential oil that is rich in compounds carrying a phenolic function” means essential oils comprising, with respect to 100% of their mass, a proportion by weight greater than or equal to 35% of compounds carrying a phenolic function.

Examples of compounds carrying a phenolic function routinely present in essential oils are thymol, carvacrol, eugenol, iso-eugenol or estragole.

Essential oils that are rich in compounds carrying a phenolic function include thyme, savory, oregano, Ceylon cinnamon, basil, or clove oil.

The hydrophobic perfuming substances of synthetic origin are obtained by chemical reaction between at least two chemical compounds, themselves of natural or synthetic origin.

Hydrophobic perfuming substances of synthetic origin include alcohols such as benzyl alcohol, 3,7-dimethyl-1-octanol, isononyl alcohol, α-terpineol, menthol, linalol, citronellol, eucalyptol or 1,8-cineol, geraniol, phytol, iso-phytol, α-terpineol, tetrahydrolinalol, farnesol, carotol, nerol, globulol, vetiverol, nerolidol, dihydromyrcenol, tetrahydromyrcenol, fenchyl alcohol, dimethyl benzyl carbinol, cinnamic alcohol, 2-phenyl ethanol or undecavertol.

Hydrophobic perfuming substances of synthetic origin include phenolic derivatives such as anethole, safrole, isosafrole, eugenol, iso-eugenol, guaiacol or 2-methoxyphenol, chavicol (or 4-allyl phenol), estragole (or 1-allyl 4-methoxy benzene), cumic alcohol, thymol or para-cresol.

In accordance with a more particular aspect, the invention pertains to the use as described above in which the hydrophobic perfuming substance (A) is selected from the group constituted by the essential oils from thyme, savory, oregano, Ceylon cinnamon, basil, cloves, bourbon geranium, Egyptian geranium, lavender aspic, rosalina, coriander, rose wood, rose petals, palmarosa, peppermint, monarda fistulosa, lavandin super or lavandin grosso.

In accordance with a particular aspect, the invention pertains to the use as described above, in which the hydrophobic perfuming substance (A) is selected from the group constituted by benzyl alcohol, 3,7-dimethyl 1-octanol, isononyl alcohol, α-terpineol, menthol, linalol, citronellol, eucalyptol (or 1,8-cineol), geraniol, phytol, iso-phytol, tetrahydrolinalol, farnesol, carotol, nerol, globulol, vetiverol, nerolidol, dihydromyrcenol, tetrahydromyrcenol, fenchyl alcohol, benzyl dimethylcarbinol, cinnamic alcohol, 2-phenyl ethanol, undecavertol, anethole, safrole, isosafrole, eugenol, iso-eugenol, guaiacol (or 2-methoxyphenol), chavicol (or 4-allyl phenol), estragole or (1-allyl 4-methoxy benzene), cumic alcohol, thymol and p-cresol.

In accordance with a particular aspect, the invention pertains to the use as described above in which in formula (I), p represents a decimal value greater than or equal to 1.05 and less than or equal to 2.5.

In accordance with a particular aspect, the invention pertains to the use as described above in which, in the definition of the compounds with formula (I), G represents the residue of a reducing sugar selected from the residues glucose, saccharose, fructose, idose, gulose, galactose, maltose, isomaltose, maltotriose, lactose, cellobiose, mannose, ribose, xylose, arabinose, lyxose, allose, altrose, rhamnose, dextran or tallose.

In accordance with a more particular aspect, the invention pertains to the use as described above, in which in formula (I), G represents the residue of a reducing sugar selected from the residues glucose, xylose and arabinose.

In accordance with a particular aspect, the invention pertains to the use as described above in which, in the definition of the compounds with formula (I), G represents the residue of a reducing sugar selected from the residues glucose, xylose and arabinose, and p represents a decimal value greater than or equal to 1.05 and less than or equal to 2.5.

In accordance with a yet more particular aspect, the invention pertains to the use as described above in which, in the definition of the compounds with formula (I), G represents the residue glucose and p represents a decimal value greater than or equal to 1.05 and less than or equal to 2.5.

In accordance with another particular aspect, the invention pertains to the use as defined above, in which the ratio by weight between said hydrophobic perfuming substance (A) and the composition represented by the formula (I) is greater than or equal to 1/7 and less than or equal to 2/1.

The invention also pertains to an aqueous solution (C₂) comprising, with respect to 100% of its mass:

a)—0.05% to 70% by weight of at least one composition represented by the formula (I):

R—O-(G)ₚ-H (I)

in which R represents the n-heptyl radical, G represents the residue of a reducing sugar and p represents a decimal value greater than or equal to 1.05 and less than or equal to 5, said composition with formula (I) consisting of a mixture of compounds represented by the formulae (I₁), (I₂), (I₃), (I₄) and (I₅):

R—O-(G)₁-H (I₁)

R—O-(G)₂-H (I₂)

R—O-(G)₃-H (I₃)

R—O-(G)₄-H (I₄)

R—O-(G)₅-H (I₅)

in the respective molar proportions a₁, a₂, a₃, a₄ and a₅, such that the sum a₁+a₂+a₃+a₄+a₅ is equal to one and such that each of the proportions a₁, a₂, a₃, a₄ and a₅ is greater than or equal to zero and less than or equal to one;

b)—0.1% to 10% by weight of at least one hydrophobic perfuming substance (A) selected from the group constituted by essential oils that are rich in compounds carrying a phenolic function, essential oils that are rich in compounds carrying an alcohol function, benzyl alcohol, 3,7-dimethyl 1-octanol, isononyl alcohol, alpha terpineol, menthol, linalol, citronellol, eucalyptol, geraniol, phytol, iso-phytol, tetrahydrolinalol, farnesol, carotol, nerol, globulol, vetiverol, nerolidol, dihydromyrcenol, tetrahydromyrcenol, fenchyl alcohol, benzyl dimethylcarbinol, cinnamic alcohol, 2-phenyl ethanol, undecavertol, anethole, safrole, isosafrole, eugenol, iso-eugenol, guaiacol, chavicol, estragole, cumic alcohol, thymol, p-cresol, and mixtures thereof;

c)—20% to 99.85% by weight of water.

In accordance with a more particular aspect, the invention pertains to an aqueous solution (C₂) as defined above comprising, with respect to 100% of its mass:

a)—0.7% to 56% by weight, more particularly 0.7% to 35% by weight of at least one composition represented by the formula (I) as defined above;

b)—0.1% to 8% by weight, more particularly 0.1% to 5% by weight of at least one hydrophobic perfuming substance (A) as selected above;
c)—36% to 99.2% by weight, more particularly 60% to 99.2% by weight of water.

In accordance with a particular aspect, the invention pertains to an aqueous solution (C₂) as defined above in which, in formula (I), p represents a decimal value greater than or equal to 1.05 and less than or equal to 2.5.

In accordance with another particular aspect, the invention pertains to an aqueous solution (C₂) as defined above in which, in formula (I), G represents the residue of a reducing sugar selected from the residues glucose, saccharose, fructose, idose, gulose, galactose, maltose, isomaltose, maltotriose, lactose, cellobiose, mannose, ribose, xylose, arabinose, lyxose, allose, altrose, rhamnose, dextran or tallose; more particularly, G represents the residue of a reducing sugar selected from the residues glucose, xylose and arabinose.

In accordance with another particular aspect, the invention pertains to an aqueous solution (C₂) as defined above in which, in the definition of the compounds with formula (I), G represents the residue of a reducing sugar selected from glucose, xylose and arabinose, and p represents a decimal value greater than or equal to 1.05 and less than or equal to 2.5.

In accordance with a more particular aspect, the invention pertains to an aqueous solution (C₂) as defined above in which, in the definition of the compounds with formula (I), G represents the glucose residue and p represents a decimal value greater than or equal to 1.05 and less than or equal to 2.5.

In accordance with a more particular aspect, the invention pertains to an aqueous solution (C₂) as defined above in which, in formula (I), G represents the residue of a reducing sugar selected from glucose, xylose and arabinose, p represents a decimal value greater than or equal to 1.05 and less than or equal to 2.5, and the hydrophobic perfuming substance (A) is selected from the group constituted by:
essential oils that are rich in compounds carrying a phenolic function, essential oils that are rich in compounds carrying an alcohol function as defined above, and
the following compounds: benzyl alcohol, 3,7-dimethyl-1-octanol, isononyl alcohol, alpha terpineol, menthol, linalol, citronellol, eucalyptol, geraniol, phytol, iso-phytol, α-terpineol, tetrahydrolinalol, farnesol, carotol, nerol, globulol, vetiverol, nerolidol, dihydromyrcenol, tetrahydromyrcenol, fenchyl alcohol, dimethyl benzyl carbinol, cinnamic alcohol, 2-phenylethanol-1, undecavertol, anethole, safrole, isosafrole, eugenol, iso-eugenol, guaiacol, chavicol, estragole, cumic alcohol, thymol and p-cresol.

In accordance with another particular aspect, the invention pertains to an aqueous solution (C₂) as defined above, in which the ratio by weight between said hydrophobic perfuming substance (A) and the composition represented by the formula (I) is greater than or equal to 1/7 and less than or equal to 2/1.

In the context of the present invention, the term “aqueous solution” means compositions (C₂) as defined above, in the form of a single-phase solution.

In accordance with a particular aspect, the invention pertains to an aqueous solution (C₂) as defined above that has a turbidity greater than or equal to 1 NTU and less than or equal to 100 NTU, more particularly greater than or equal to 1 NTU and less than or equal to 50 NTU, and still more particularly greater than or equal to 1 NTU and less than or equal to 25 NTU, said turbidity being measured quantitatively using an optical nephelometric method, in particular using an optical turbidimeter from the HF Scientific range with model number DRT100B.

The aqueous solution (C₂) in accordance with the invention is prepared by mixing its constituents, with mechanical stirring, at a stirring rate in the range 50 revolutions per minute to 500 revolutions per minute, more particularly in the range 50 revolutions per minute to 100 revolutions per minute, at a temperature in the range 20° C. to 80° C., more particularly in the range 20° C. to 60° C., and still more particularly in the range 20° C. to 45° C.

The aqueous solution (C₂) as defined above may also comprise ingredients and additives that are in normal use in the perfumery field and in perfuming compositions, such as volatile solvents, hydrosoluble antioxidants, hydrosoluble sequestrating agents, hydrosoluble colorants, color-stabilizing agents, peptizing agents, hydrosoluble hydrating agents, hydrosoluble vitamins, and propellants.

Volatile solvents that may be associated with the aqueous solution (C₂) in accordance with the invention include hydrosoluble and volatile alcohols such as ethanol, isopropanol or butanol, and more particularly ethanol, organic solvents such as glycerol, diglycerol, glycerol oligomers, ethylene glycol, propylene glycol, butylene glycol, 1,3-propanediol, 1,2-propanediol, hexylene glycol, diethylene glycol, xylitol, erythritol, or sorbitol.

In accordance with a particular aspect, the composition (C₂) in accordance with the invention comprises, with respect to 100% of its mass, up to 20% by weight of one or more volatile solvents selected from ethanol, isopropanol or n-butanol.

In accordance with another particular aspect, the composition (C₂) in accordance with the invention does not comprise volatile solvents.

Hydrosoluble antioxidants that may be associated with the aqueous solution (C₂) in accordance with the invention include ascorbic acid, glutathione, tartaric acid, oxalic acid, and tetrasodium glutamate diacetate.

Hydrosoluble sequestrating agents that may be associated with the aqueous solution (C₂) in accordance with the invention include ethylene diamine tetra acetic acid salts (EDTA), such as the EDTA sodium salt, and salts of diethylene triamine pentaacetic acid (DTPA) such as DTPA sodium salts.

Hydrosoluble colorants that may be associated with the aqueous solution (C₂) in accordance with the invention include caramel, Yellow 5, Acid Blue 9/Blue 1, Green 5, Green 3/Fast Green FCF 3, Orange 4, Red 4/Food Red 1, Yellow 6, Acid Red 33/Food Red 12, Red 40, crimson lake (CI 15850, CI 75470), Ext. Violet 2, Red 6-7, Ferric Ferrocyanide, Ultramarines, Acid Yellow 3/Yellow 10, Acid Blue 3, and Yellow 10.

Color-stabilizing hydrosoluble agents that may be associated with the aqueous solution (C₂) in accordance with the invention include tris(tetramethylhydroxy piperidinol) citrate, sodium benzotriazolyl butylphenol sulfonate, and benzotriazolyl dodecyl p-cresol.

Agents that can improve the clarity and/or reduce cold precipitation phenomena that may be associated with the aqueous solution (C₂) in accordance with the invention include cellulose alkylethers such as cellulose methylether, cellulose ethylether, or cellulose methyl ethyl ether.

Hydrosoluble hydrating agents that may be associated with the aqueous solution (C₂) in accordance with the invention include glycerol, glycerol oligomers such as diglycerol, triglycerol, urea, hydroxyureas, glycerol glucoside, diglycerol glucoside, polyglyceryl glucosides, erythrityl glucoside, sorbityl glucoside, xylityl glucoside, the composition marketed under the trade name AQUAXYL™ comprising xylityl glucoside, anhydroxylitol and xyllitol.

When it is in the form of a lotion, the aqueous solution (C₂) in accordance with the invention as defined above may be applied as a cologne, as an eau de toilette, as an eau de parfum, or as an aftershave lotion.

When it is packaged into bottles, the aqueous solution (C₂) in accordance with the invention as defined above may be applied in the form of fine droplets using mechanical pressurization devices or using a propellant gas.

Propellants that may be associated with the aqueous solution (C₂) in accordance with the invention include hydrofluorinated compounds such as dichlorodifluoromethane, trichlorofluoromethane, difluorethane, isobutane, n-butane or propane.

The invention also pertains to a method for perfuming the skin, the hair, the scalp, the mucous membranes or clothing, comprising a step for application to said skin, said hair, said scalp, said mucous membranes or said clothing of the aqueous solution (C₂) as defined above.

The invention also pertains to the use of the aqueous solution (C₂) as defined above as a perfuming agent for the preparation of a cosmetic composition (C₃) for topical use.

The term “perfuming agent”, means the action by said agent of providing an odor, which persists to a greater or lesser extent, to a cosmetic composition (C₃) for topical use.

The expression “for topical use” means that the composition (C₃) in accordance with the invention is employed by application to the skin, the hair or the mucous membranes, irrespective of whether it concerns direct application or indirect application when, for example, the composition (C₃) in accordance with the invention is incorporated into a support intended to come into contact with the skin (paper, towellette, textile, transdermal device, etc).

The cosmetic composition (C₃) for topical use may be in the form of an aqueous gel, an emulsion or a microemulsion which may be of the oil-in-water type (O/W), water-in-oil (W/O), oil-in-water-in-oil (O/W/O), or water-in-oil-in-water (W/O/W) type. The oily phase of the emulsion or the microemulsion may consist of a mixture of one or more oils.

When the cosmetic composition (C₃) for topical use is in the form of an aqueous gel (AG), said aqueous gel (AG) comprises an effective quantity of at least one thickening agent and/or gelling agent.

The term “effective quantity of at least one thickening agent and/or gelling agent” means a quantity by weight such that it can be used to increase the dynamic viscosity of the aqueous solution (C₂) as defined above to the desired value. The effective quantity of at least one thickening agent and/or gelling agent, with respect to 100% of the mass of said cosmetic composition (C₃) for topical use in the form of an aqueous gel (AG), is generally greater than or equal to 0.05% by weight and less than or equal to 5% by weight, more particularly greater than or equal to 0.1% by weight and less than or equal to 5% by weight, and still more particularly greater than or equal to 0.1% by weight and less than or equal to 3% by weight.

In general, the dynamic viscosity of the cosmetic composition (C₃) for topical use in the form of an aqueous gel (AG) measured at 20° C. using a Brookfield LV type viscosimeter at a speed of 5 revolutions per minute with a suitable rotor, is greater than or equal to 5 000 mPa·s and less than or equal to 200 000 mPa·s, more particularly greater than or equal to 5 000 mPa·s and less than or equal to 100 000 mPa·s, and still more particularly greater than or equal to 10 000 mPa·s and less than or equal to 80 000 mPa·s.

In accordance with a particular aspect, the thickening and/or gelling agent included in the cosmetic composition (C₃) for topical use in the form of an aqueous gel (AG) is selected from polysaccharides constituted solely by oses.

In accordance with a more particular aspect, the thickening and/or gelling agent comprised in the cosmetic composition (C₃) for topical use in the form of an aqueous gel (AG) is selected from polysaccharides constituted solely by oses selected from the elements of the group constituted by glucans or glucose homopolymers, glucomannoglucans, xyloglycans, galactomannans wherein the degree of substitution (DS) of the D-galactose units on the principal chain of the D-mannose is in the range 0 to 1, and more particularly in the range 0.25 to 1, such as galactomannans from cassia gum (DS=1/5), carouba gum (DS=1/4), tara gum (DS=1/3), guar gum (DS=1/2), and fenugreek gum (DS=1).

In accordance with a yet more particular aspect, the thickening and/or gelling agent comprised in the cosmetic composition (C₃) for topical use in the form of an aqueous gel (AG) is selected from polysaccharides constituted solely by oses selected from the elements of the group constituted by galactomannans deriving from carouba gum (DS=1/4), tara gum (DS=1/3), and guar gum (DS=1/2).

In accordance with another particular aspect, the thickening and/or gelling agent comprised in the cosmetic composition (C₃) for topical use in the form of an aqueous gel (AG) is selected from polysaccharides constituted by derivatives of oses.

In accordance with a more particular aspect, the thickening and/or gelling agent comprised in the cosmetic composition (C₃) for topical use in the form of an aqueous gel (AG) is selected from polysaccharides constituted by derivatives of oses selected from the elements of the group constituted by sulfated galactans, and more particularly carrageenans and agar, uronans and more particularly algins, alginates and pectins, heteropolymers of oses and of uronic acids, and more particularly xanthan gum, gellan gum, exudates of gum Arabic and karaya gum, and glucosaminoglycans.

In accordance with a yet more particular aspect, the thickening and/or gelling agent comprised in the cosmetic composition (C₃) for topical use in the form of an aqueous gel (AG) is selected from polysaccharides constituted by derivatives of oses selected from the elements of the group constituted by alginates, xanthan gum, and exudates of gum Arabic.

In accordance with a particular aspect, the thickening and/or gelling agent comprised in the cosmetic composition (C₃) for topical use in the form of an aqueous gel (AG) is selected from cellulose, cellulose derivatives such as methyl cellulose, ethyl cellulose, hydroxypropyl cellulose, silicates, starch, hydrophilic starch derivatives, and polyurethanes.

In accordance with a particular aspect, the thickening and/or gelling agent comprised in the cosmetic composition (C₃) for topical use in the form of an aqueous gel (AG) is selected from linear or branched and/or cross-linked polyelectrolyte type polymers.

In accordance with a more particular aspect, the thickening and/or gelling agent comprised in the cosmetic composition (C₃) for topical use in the form of an aqueous gel (AG) is selected from the following group constituted by linear, branched and/or cross-linked polyelectrolyte type polymers:
homopolymers of acrylic acid, completely or partially in the salt form, of methacrylic acid, completely or partially in the salt form, or of 2-methyl [(1-oxo-2-propenyl) amino] 1-propanesulfonic acid (AMPS), completely or partially in the salt form;
the copolymer of acrylic acid or methacrylic acid, completely or partially in the salt form, and 2-methyl [(1-oxo-2-propenyl) amino] 1-propanesulfonic acid (AMPS), completely or partially in the salt form;
the copolymers of acrylic acid or methacrylic acid, completely or partially in the salt form, and of a neutral polymer selected from acrylamide, (2-hydroxyethyl) acrylate, N,N-dialkyl acrylamide such as N,N-dimethyl acrylamide, N-vinyl pyrrolidone, (2-hydroxyethyl) methacrylate, tris(hydroxymethyl) acrylamidomethane (THAM) and N-(2-hydroxyethyl) acrylamide;
the copolymers of 2-methyl [(1-oxo-2-propenyl) amino] 1-propanesulfonic acid (AMPS), completely or partially in the salt form, and of a neutral monomer selected from acrylamide, (2-hydroxyethyl) acrylate, N,N-dialkyl acrylamide such as N,N-dimethyl acrylamide, N-vinyl pyrrolidone, (2-hydroxyethyl) methacrylate, tris(hydroxymethyl) acrylamidomethane (THAM) and N-(2-hydroxyethyl) acrylamide;
the terpolymers of 2-methyl [(1-oxo-2-propenyl) amino] 1-propanesulfonic acid (AMPS), completely or partially in the salt form, acrylic or methacrylic acid, completely or partially in the salt form, and of a neutral monomer selected from acrylamide, (2-hydroxyethyl) acrylate, N,N-dialkyl acrylamide such as N,N-dimethyl acrylamide, N-vinyl pyrrolidone, (2-hydroxyethyl) methacrylate, tris(hydroxy methyl) acrylamidomethane (THAM) and N-(2-hydroxyethyl) acrylamide;
the terpolymers of 2-methyl-[(1-oxo-2-propenyl)amino] 1-propanesulfonic acid (AMPS), completely or partially in the salt form, of acrylic acid, completely or partially in the salt form, and of methacrylic acid, completely or partially in the salt form;
the copolymers of acrylic acid or methacrylic acid and alkyl acrylates, wherein the carbon chain contains between four and thirty carbon atoms and more particularly between ten and thirty carbon atoms;
the copolymers of 2-methyl [(1-oxo-2-propenyl) amino] 1-propanesulfonic acid (AMPS), completely or partially in the salt form, and of alkyl acrylates or methacrylates wherein the carbon chain contains between four and thirty carbon atoms and more particularly between ten and thirty carbon atoms;
polymers, and more particularly cross-linked copolymers, terpolymers and tetrapolymers comprising 2-methyl [(1-oxo-2-propenyl)amino] 1-propanesulfonic acid (AMPS), completely or partially in the salt form, and macromers, such as those described and claimed in the European patent application published with the number EP 1 069 142 A1, in the European patent application published with the number EP 1 339 789 A2, in the international application published with the number WO 2011/030044 A1 and in the French patent application published with the number FR 2 910 899 A1.

When the cosmetic composition (C₃) for topical use is in the form of an emulsion (E₁), said emulsion (E₁) comprises a fatty phase (P₁) constituted by at least one oil and/or at least one wax, and an emulsification system (S₁) comprising at least one emulsifying surfactant.

The term “oils” as used in the present patent application means compounds and/or mixtures of compounds which are insoluble in water, having a liquid appearance at a temperature of 25° C. Oils which may constitute the fatty phase (P₁) comprised in the cosmetic composition (C₃) for topical use in the form of an emulsion (E₁) include:
mineral oils such as paraffin oil, vaseline oil, iso paraffins or white mineral oils;
oils of animal origin such as squalene or squalane;
vegetable oils such as phytosqualane, karite butter or sweet almond, coprah, castor, jojoba, olive, rape, peanut, sunflower, wheatgerm, corn, soya, cotton, alfalfa, poppy, squash, evening primrose, millet, barley, rye, safflower, candle nut, passion flower, hazelnut, palm, apricot kernel, calophyllum, sisymbrium, avocado, calendula or indeed those obtained from petals or legumes;
ethoxylated vegetable oils;
synthetic oils such as:
esters of fatty acids such as butyl, propyl or cetyl myristates, isopropyl palmitate, butyl, hexadecyl, isopropyl, octyl or isocetyl stearates, dodecyl oleate, hexyl laurate or propylene glycol dicaprylate;
esters of lanolic acid, such as isopropyl or isocetyl lanolates;
monoglycerides, diglycerides and triglycerides of fatty acids, such as glycerol triheptanoate; or indeed
alkylbenzoates, hydrogenated oils, poly(α-olefins), polyolefins such as poly(isobutane), synthesized iso-alkanes such as isohexadecane, isododecane, perfluorinated oils and silicone oils such as dimethylpolysiloxanes, methylphenylpolysiloxanes, silicones modified by amines, silicones modified by fatty acids, silicones modified by alcohols, silicones modified by alcohols and fatty acids, silicones modified by polyether groups, epoxy-modified silicones, silicones modified by fluorine groups, cyclic silicones and silicones modified by alkyl groups.

The term “waxes” as used in the present patent application means compounds and/or mixtures of compounds which are insouble in water, and which are solid in appearance at a temperature of 45° C. or above. Waxes which could constitute the fatty phase (P₁) comprised in the cosmetic composition (C₃) for topical use in the form of an emulsion (E₁) include beeswax, carnauba wax, candelilla wax, ouricury wax, Japan wax, cork fiber wax, sugar cane wax, paraffin wax, lignite wax, lanolin, polyethylene or silicone wax or microcrystalline waxes, plant waxes, ozokerite, fatty alcohols and fatty acids that are solids at ambient temperature, or glycerides that are solids at ambient temperature.

Other fats that may be associated with the fatty phase (P₁) comprised in the cosmetic composition (C₃) for topical use in the form of an emulsion (E₁) include saturated or unsaturated, linear or branched fatty alcohols or saturated or unsaturated, linear or branched fatty acids.

Examples of emulsifying surfactants which could constitute the emulsification system (S₁) comprised in the cosmetic composition (C₃) for topical use in the form of an emulsion (E₁) include emulsifying surfactants selected amphoteric, anionic, cationic or non-ionic surfactants; the emulsifying surfactants are selected as a function of the type of emulsion (E₁), namely an emulsion (E₁₁) of the water-in-oil type (W/O) or an emulsion (E₁₂) of the oil-in-water type (O/W).

Examples of emulsifying surfactants which could constitute the emulsification system (S₁) comprised in the cosmetic composition (C₃) for topical use in the form of an emulsion (E₁₁) of the water-in-oil type include:
ethoxylated fatty alcohols, wherein the fatty alkyl chain, which may be saturated or unsaturated, linear or branched, contains eight to thirty-six carbon atoms and more particularly twelve to twenty-two carbon atoms, and wherein the degree of ethoxylation is in the range 1 to 5, and more particularly in the range 1 to 3;
fatty and ethoxylated acids, wherein the fatty alkyl chain, which may be saturated or unsaturated, linear or branched, contains eight to thirty-six carbon atoms and more particularly twelve to twenty-two carbon atoms, and wherein the degree of ethoxylation is in the range 1 and 5, and more particularly in the range 1 to 3;
esters of vegetable oils such as those of rapeseed oil, sunflower oil or corn oil;
ethoxylated esters of vegetable oils, such as the ethoxylated methyl ester of rapeseed oil containing a quantity of ethylene oxide in the range 1 to 5 moles, more particularly in the range 1 to 3 moles;
fatty glycerol esters wherein the fatty alkyl chain, which may be saturated or unsaturated, linear or branched, contains eight to thirty-six carbon atoms and more particularly ten to twenty-two carbon atoms and still more particularly twelve to eighteen carbon atoms, such as glycerol stearate, glycerol oleate, glycerol palmitate or glycerol isosterate;
fatty oligoglycerol esters wherein the fatty alkyl chain, which may be saturated or unsaturated, linear or branched, contains eight to twenty-two carbon atoms, and wherein the number of moles of glycerol is in the range 2 to 10, such as tetraglycerol, decaglycerol, or diglycerol isostearates, diglycerol laurate, diglycerol or tetraglycerol palmitates, decaglycerol oleate, decaglycerol monolaurate, decaglycerol mono-linoleate or decaglycerol mono-myristate;
fatty sorbitan esters wherein the fatty alkyl chain, which may be saturated or unsaturated, linear or branched, contains eight to eighteen carbon atoms, and more particularly twelve to eighteen carbon atoms, such as sorbitan monooleate, sorbitan dioleate, sorbitan trioleate, sorbitan tetraoleate, sorbitan monostearate, sorbitan distearate, sorbitan tristearate, sorbitan tetrastearate, sorbitan monoisostearate, sorbitan diisostearate, sorbitan triisostearate, sorbitan tetraisostearate, sorbitan monopalmitate, sorbitan dipalmitate, sorbitan tripalmitate, sorbitan tetrapalmitate, sorbitan monolaurate, sorbitan dilaurate, sorbitan trilaurate or sorbitan tetralaurate;
alkyl polyglycoside compositions, those of alkyl polyglucosides, such as isostearyl polyglucosides, of alkyl polyxylosides, such as those of isostearyl xylosides or octyl-2 dodecyl polyxyloside compositions;
compositions of alkyl polyglycosides and fatty alcohols, such as those of alkyl polyglucosides and fatty alcohols, for example those of isostearyl polyglucosides and isostearyl alcohol; or compositions of alkyl polyxylosides and fatty alcohols, such as those of isostearyl xylosides and isostearyl alcohol or those of octyl-2 dodecyl polyxylosides and 2-octyldodecyl marketed under the trade name FLUIDANOV™20X;
polyglycol polyhydroxystearates represented by the formula (II):

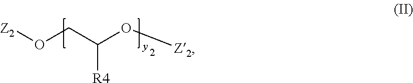

in which y₂ represents a whole number greater than or equal to 2 and less than or equal to 50, R₄ represents the hydrogen atom, the methyl radical or the ethyl radical, Z₂ represents a radical with formula (III):

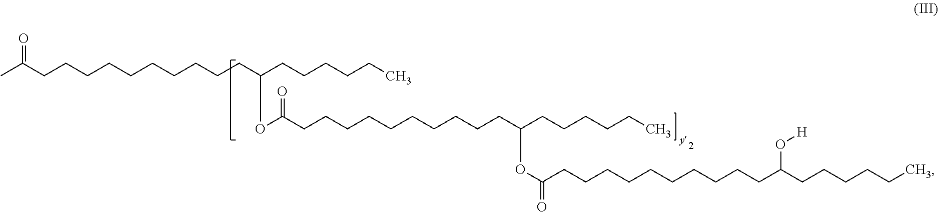

in which y′₂ represents a whole number greater than or equal to 0 and less than or equal to 10, more particularly greater than or equal to 1 and less than or equal to 10; and Z′₂ represents a radical with formula (III) as defined above, with Z₂′ being identical to or different from Z₂, or the hydrogen atom.
polyglycol polyhydroxystearates represented by the formula (IV):

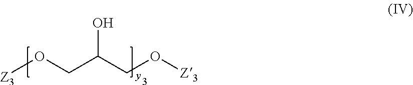

in which Z₃ represents a radical with formula (III) as defined above, Z′₃ represents a radical with formula (III) as defined above, with Z₃′ being identical to or different from Z₃, or the hydrogen atom, and y₃ represents a whole number greater than or equal to 2 and less than or equal to 20.
alkoxylated polyglycerol polyhydroxystearates represented by the formula (V):

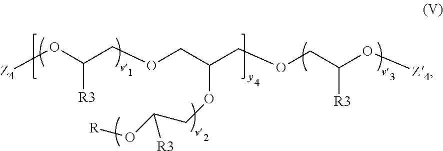

in which Z₄ represents a radical with formula (III) as defined above, Z′₄ represents a radical with formula (III) as defined above, with Z₄′ being identical to or different from Z₄, or the hydrogen atom, y₄ represents a whole number greater than or equal to 2 and less than or equal to 20, v′₁, v′₂, v′₃, which may be identical or different, represent a whole number greater than or equal to 0 and less than or equal to 50, and the sum [(y₄. V′₁)+(y₄. V′₂)+V′₃)] is greater than or equal to 1 and less than or equal to 50;
polyethyleneglycol-alkylglycol copolymers represented by the formula (VI):

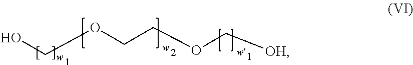

in which w₁ and w′₁, which may be identical or different, represent a whole number greater than or equal to 1 and less than or equal to 50, more particularly greater than or equal to 1 and less than or equal to 25, w₂ represents a whole number greater than or equal to 1 and less than or equal to 100, more particularly greater than or equal to 1 and less than or equal to 50.

Examples of emulsifying surfactants which could constitute the emulsification system (S₁) comprised in the cosmetic composition (C₃) for topical use in the form of an emulsion (E₁₂) of the oil-in-water type include:
fatty and ethoxylated alcohols, wherein the fatty alkyl chain, which may be saturated or unsaturated, linear or branched, contains sixteen to thirty-six carbon atoms and more particularly twelve to twenty-two carbon atoms, and wherein the degree of ethoxylation is greater than 5 and less than or equal to 200, and more particularly greater than or equal to 10 and less than or equal to 100, such as decaethoxylated oleodecyl alcohols, with the trade name SIMULSOL™ OC 710, or heptaethoxylated lauric alcohols marketed with the trade name SIMULSOL™ P7;
ethoxylated and fatty acids, wherein the fatty alkyl chain, which may be saturated or unsaturated, linear or branched, contains eight to thirty-six carbon atoms and more particularly eight to twenty-two carbon atoms, and wherein the degree of ethoxylation is greater than 5 and less than or equal to 200, and more particularly greater than or equal to and less than or equal to 100;
ethoxylated vegetable oils with a quantity of ethylene oxide of more than 5 moles and less than or equal to 100 moles, more particularly greater than or equal to 10 moles and less than or equal to 50 moles, such as ethoxylated rapeseed oil containing 10 moles of ethylene oxide and marketed with the trade name SEPITER™C100, ethoxylated rapeseed oil containing 20 moles of ethylene oxide and marketed with the trade name SEPITER™C200, ethoxylated sunflower oil containing 40 moles of ethylene oxide and marketed with the trade name SEPITER™T400, ethoxylated apricot kernel oil containing 40 moles of ethylene oxide and marketed with the trade name SIMULSOL™AO40, or polyethoxylated castor oil containing 40 moles of ethylene oxide marketed with the trade name SIMULSOL™ OL50;
compositions comprising glycerol stearate and ethoxylated stearic acid containing between 5 moles and 150 moles of ethylene oxide, such as the composition comprising ethoxylated stearic acid containing 135 moles of ethylene oxide and glycerol stearate, marketed with the trade name SIMULSOL™ 165;
fatty ethoxylated sorbitan esters, wherein the fatty alkyl chain, which may be saturated or unsaturated, linear or branched, contains eight to eighteen carbon atoms, and more particularly twelve to eighteen carbon atoms, and wherein the degree of ethoxylation is greater than or equal to 5 and less than or equal to 50, more particularly greater than or equal to 10 and less than or equal to 25, and still more particularly equal to 20, such as the products marketed with the trade name MONTANOX™ by SEPPIC,
mixtures of alkyl polyglycosides and fatty alcohols, such as those described in French patent applications 2 668 080, 2 734 496, 2 756 195, 2 762 317, 2 784 680, 2 784 904, 2 791 565, 2 790 977, 2 807 435, 2 804 432, 2 830 774, 2 830 445, and marketed with the trade names MONTANOV™ 68, MONTANOV™ 82, MONTANOV™ 202, MONTANOV™ L and MONTANOV™ 14.

When the cosmetic composition (C₃) for topical use is in the form of an emulsion (E₁), said emulsion (E₁) also comprises excipients and/or active principles that are in normal use in the field of formulations for topical use, in particular cosmetics, dermocosmetics, pharmaceuticals or dermopharmaceuticals, such as foaming surfactants and/or detergents, thickening surfactants and/or gelling agents, thickening agents and/or gelling agents, stabilizing agents, film-forming compounds, opacifying agents, pearlescent agents, superfatting agents, sequestrating agents, preservatives, conditioning agents, deodorizing agents, bleaching agents to decolorize hair and skin, active principles intended to provide a treatment action and/or protective action as regards the skin or hair, sunscreens, mineral fillers or pigments, particles providing a visual effect or intended to encapsulate active ingredients, exfoliating particles, texturizing agents, optical brighteners, and insect repellants.

Examples of foaming surfactants and/or detergents which could be present in the composition (C₃) for topical use in the form of an emulsion (E₁) include topically acceptable anionic, cationic, amphoteric or non-ionic foaming surfactants and/or detergents that are in normal use in this field of activity.

Examples of foaming surfactants and/or detergents which could be associated with the composition (C₃) for topical use in the form of an emulsion (E₁) are alkaline metal salts, alkaline-earth metal salts, ammonium salts, amine salts, salts of aminoalcohols of alkylether sulfates, of alkylsulfates, of alkylamidoethersulfates, of alkylarylpolyethersulfates, of monoglyceride sulfates, of α-olefin sulfonates, of paraffin sulfonates, of alkylphosphates, of alkyletherphosphates, of alkylsulfonates, of alkylamidesulfonates, of alkylarylsulfonates, of alkylcarboxylates, of alkylsulfosuccinates, of alkylethersulfosuccinates, of alkylamidesulfosuccinates, of alkylsulfoacetates, of alkylsarcosinates, of acylisethionates, of Nacyltaurates, of acyllactylates, of N-acylated amino acid derivatives, of N-acylated peptide derivatives, of N-acylated protein derivatives, and of fatty acids.

Examples of amphoteric foaming surfactants and/or detergents that may be associated with the composition (C₃) for topical use in the form of an emulsion (E₁) are alkylbetaines, alkylamidobetaines, sultaines, alkylamidoalkylsulfobetaines, imidazoline derivatives, phosphobetaines, amphopolyacetates and amphopropionates.

Particular examples of cationic foaming surfactants and/or detergents that may be associated with the composition (C₃) for topical use in the form of an emulsion (E₁) are quaternary ammonium derivatives.

More particular examples of non-ionic foaming surfactants and/or detergents that may be associated with the composition (C₃) for topical use in the form of an emulsion (E₁) are alkyl polyglycosides containing a linear or branched, saturated or unsaturated aliphatic radical containing 8 to 12 carbon atoms, coprah amides, and N-alkylamines.

Examples of thickening and/or gelling surfactants that may be associated with the composition (C₃) for topical use in the form of an emulsion (E₁) include the fatty esters of alkyl polyglycosides which may optionally be alkoxylated, and more particularly ethoxylated esters of methylpolyglucoside such as PEG 120 methyl glucose trioleate and PEG 120 methyl glucose dioleate respectively marketed with the trade names GLUCAMATE™ LT and GLUMATE™ DOE120; alkoxylated fatty esters such as PEG 150 pentaerythrytyl tetrastearate marketed with the trade name CROTHIX™ DS53, PEG 55 propylene glycol oleate marketed with the trade name ANTIL™ 141; polyalkylene glycol carbamates containing fatty chains, such as PPG 14 laureth isophoryl dicarbamate marketed with the trade name ELFACOS™ T211, and PPG 14 palmeth 60 hexyl dicarbamate marketed with the trade name ELFACOS™ GT2125.

Examples of thickening and/ot gelling agents that may be associated with the composition (C₃) for topical use in the form of an emulsion (E₁) include the thickening and/or gelling agents described above for the preparation of a cosmetic composition (C₃) for topical use in the form of an aqueous gel (AG).

Examples of opacifying and/or pearlescent agents that may be associated with the composition (C₃) for topical use in the form of an emulsion (E₁) include sodium palmitate, sodium stearate, sodium hydroxystearate, magnesium palmitate, magnesium stearate, magnesium hydroxystearate, ethylene glycol monostearate, ethylene glycol distearate, polyethylene glycol monostearate, polyethylene glycol distearate, and fatty alcohols containing 12 to 22 carbon atoms.

Examples of the active principles that may be associated with the composition (C₃) for topical use in the form of an emulsion (E₁) include: vitamins and derivatives thereof, especially their esters, such as retinol (vitamin A) and its esters (retinyl palmitate for example), ascorbic acid (vitamin C) and its esters, sugar derivatives of ascorbic acid (such as ascorbyl glucoside), tocopherol (vitamin E) and its esters (such as tocopherol acetate), vitamins B3 or B10 (niacinamide and its derivatives); compounds exhibiting a skin lightening or depigmenting action such as SEPIWHITE™MSH, arbutin, kojic acid, hydroquinone, VEGEWHITE™, GATULINE™, SYNERLIGHT™, BIOWHITE™ PHYTOLIGHT™, DERMALIGHT™, CLARISKIN™, MELASLOW™, DERMAWHITE™ ETHIOLINE, MELAREST™, GIGAWHITE™, ALBATINE™, LUMISKIN™; compounds exhibiting a soothing action such as SEPICALM™ S, allantoin and bisabolol; antiinflammatory agents; compounds exhibiting a hydrating action such as urea, hydroxyureas, glycerol, polyglycerols, glycerol glucoside, diglycerol glucoside, polyglyceryl glucosides; plant extracts rich in polyphenols such as grape extracts, pine extracts, vine extracts or olive extracts; compounds exhibiting a slimming or lipolytic action such as caffeine or its derivatives, ADIPOSLIM™, ADIPOLESS™; N-acylated proteins; N-acylated peptides such as MATRIXIL™; N-acylated amino acids; partial hydrolysates of N-acylated proteins; amino acids; peptides; total protein hydrolysates; soya extracts, for example Raffermine™; wheat extracts, for example TENSINE™ or GLIADINE™; tannin-rich plant extracts; isoflavone-rich plant extracts; terpene-rich plant extracts; fresh water algae extracts, sea water algae extracts, brackish water algae extracts; marine halophyte plant extracts; marine extracts in general such as corals; bacterial extracts; ceramides; phospholipids; compounds exhibiting an antimicrobial action or a purifying action, such as LIPACIDE™ C8G, LIPACIDE™ UG, SEPICONTROL™ A5; OCTOPIROX™ or SENSIVA™ SC50; compounds exhibiting an energizing or stimulating property, such as Physiogenyl™, panthenol and its derivatives such as SEPICAP™ MP; anti-aging active ingredients such as SEPILIFT™ DPHP, LIPACIDE™ PVB, SEPIVINOL™, SEPIVITAL™ MANOLIVA™, PHYTO-AGE™, TIMECODE™; or SURVICODE™; anti-photo-aging active ingredients; active ingredients protecting the integrity of the dermo-epidermal junction; active ingredients enhancing the synthesis of components of the extracellular matrix, such as collagen, elastins, glycosaminoglycans; active ingredients that promote chemical cell communication, such as cytokines, or physical communication, such as integrins; active ingredients creating a “heating” sensation on the skin, such as activators of cutaneous microcirculation (such as nicotinic acid derivatives) or products creating a sensation of “freshness” on the skin (such as menthol and its derivatives); active ingredients improving cutaneous microcirculation, for example veinotonics; draining agents; decongestant active ingredients such as extracts of ginko biloba, ivy, horse chestnut, bamboo, ruscus, butcher's broom, centalla asiatica, fucus, rosemary, willow; skin darkening or tanning agents, such as dihydroxyacetone, isatin, alloxan, ninhydrin, glyceraldehyde, mesotartaric aldehyde, glutaraldehyde, or erythrulose.

Examples of deodorizing agents that may be associated with the composition (C₃) for topical use in the form of an emulsion (E₁) include alkaline silicates, zinc salts such as zinc sulfate, zinc gluconate, zinc chloride, or zinc lactate; quaternary ammonium salts such as cetyltrimethylammonium salts, or cetylpyridinium salts; glycerol derivatives such as glycerol caprate, glycerol caprylate, or polyglycerol caprate; 1,2 decanediol; 1,3 propanediol; salicylic acid; sodium bicarbonate; cyclodextrins; metallic zeolites; Triclosan™; aluminum hydrobromide, aluminum hydrochlorides, aluminum chloride, aluminum sulfate, aluminum and zirconium hydrochlorides, aluminum and zirconium trihydrochloride, aluminum and zirconium tetrahydrochloride, aluminum and zirconium pentahydrochloride, aluminum and zirconium octahydrochloride, aluminum sulfate, sodium and aluminum lactate, complexes of aluminum hydrochloride and glycol such as the complex of aluminum hydrochloride and propylene glycol, the complex of aluminum dihydrochloride and propylene glycol, the complex of aluminum sesquihydrochloride and propylene glycol, the complex of aluminum hydrochloride and polyethylene glycol, the complex of aluminum dihydrochloride and polyethylene glycol, or the complex of aluminum sesquihydrochloride and polyethylene glycol.

Examples of sunscreens that may be associated with the composition (C₃) for topical use in the form of an emulsion (E₁) are any of those listed in the Annex VII of the modified cosmetics directive 76/768/EEC.

When the cosmetic composition (C₃) for topical use is in the form of an emulsion (E₁₂) of the oil-in-water type, said emulsion (E₁₂) generally comprises:
5% to 55% by weight, more particularly 7% to 30% by weight, and still more particularly 10% to 20% by weight of a fatty phase (P1) constituted by at least one oil and/or at least one wax as described above,
95% to 45% by weight, more particularly 93% to 70% by weight, and still more particularly 90% to 80% by weight of the aqueous solution (C₂) as defined above.

When the cosmetic composition (C₃) for topical use is in the form of an emulsion (E₁₁) of the water-in-oil type, said emulsion (E₁₁) generally comprises:
5% to 70% by weight, more particularly 10% to 60% by weight, and still more particularly 20% to 60% by weight of the aqueous solution (C₂) as defined above,
95% to 30% by weight, more particularly 90% to 40% by weight, and still more particularly 80% to 40% by weight of a fatty phase (P1) constituted by at least one oil and/or at least one wax as described above.

The experimental study below illustrates the invention without in any way limiting its scope.

1) Preparation of Compositions Represented by the Formula (I) and Evaluation of their Solubilizing Properties in Accordance with the Invention
1.1 Preparation of a Composition of n-Heptyl Polyglucosides

2.7 molar equivalents of n-heptanol were introduced into a jacketed glass reactor in which a heat transfer fluid was circulated, provided with efficient stirring, at a temperature of 40° C. One molar equivalent of anhydrous glucose was then slowly added to the reaction medium to enable it to be dispersed homogeneously, then 0.15% by weight of 98% sulfuric acid and 0.15% by weight of 50% hypophosphorous acid with respect to 100% of the mass constituted by the sum of the mass of glucose and the mass of n-heptanol were introduced into the previously prepared homogeneous dispersion. The reaction medium was placed under a partial vacuum of approximately 180 mbar and maintained at a temperature of 100° C.-105° C. for a period of 4 hours, evacuating the water formed using a distillation apparatus. The reaction medium was then cooled to 85° C.-90° C. and neutralized by adding 40% sodium hydroxide to bring the pH of a 5% solution of this mixture to a value of approximately 7.0. The reaction medium obtained thereby was then evacuated at a temperature of 70° C. and filtered to eliminate the unreacted grains of glucose. The filtrate was then introduced into a jacketed glass reactor in which a heat transfer fluid was circulated, provided with efficient stirring and a distillation apparatus. The excess heptanol was then eliminated by distillation at a temperature of 120° C. under a partial vacuum of between approximately 100 mbar and 50 mbar. The distilled reaction medium was immediately diluted by adding a quantity of water in a manner such as to obtain a concentration of reaction medium of approximately 60%. After homogenization for 30 minutes at a temperature of 50° C., the composition (X₁) obtained was evacuated.

The analytical characteristics of the composition (X₁) obtained thereby comprising n-heptyl polyglucosides are presented in Table 1 below.

TABLE 1

| | Composition (X₁) |
|---|---|
| Appearance at 20° C. (Visual determination) | Liquid |
| Acid number (NFT standard 60204) | 1.7 |
| Dry extract hydroxyl value (USP XXI NF XVI | 813.9 |
| standard Jan. 1, 1995) | |
| Water (% by weight) (NFT standard 73201) | 58.8% |
| Residual n-heptanol content (gas phase | 0.22% |
| chromatography), as a % by weight | |

### 2) Preparation of Comparative Compositions

2.1 Preparation of a Composition of n-Pentyl Polyglucosides

3.1 molar equivalents of n-pentanol were introduced into a jacketed glass reactor in which a heat transfer fluid was circulated, provided with efficient stirring, at a temperature of 105° C. One molar equivalent of anhydrous glucose was then slowly added to the reaction medium to enable it to be dispersed homogeneously, then 0.14% by weight of 98% sulfuric acid and 0.14% by weight of 50% hypophosphorous acid with respect to 100% of the mass constituted by the sum of the mass of glucose and the mass of n-pentanol were introduced into the previously prepared homogeneous dispersion. The reaction medium was placed under a partial vacuum of approximately 400 mbar and maintained at a temperature of 100° C.-105° C. for a period of 6 hours, evacuating the water formed using a distillation apparatus. The reaction medium was then cooled to 85° C.-90° C. and neutralized by adding 40% sodium hydroxide to bring the pH of a 5% solution of this mixture to a value of approximately 7.0. The reaction medium obtained thereby was then evacuated at a temperature of 70° C. and filtered to eliminate the unreacted grains of glucose. The filtrate was then introduced into a jacketed glass reactor in which a heat transfer fluid was circulated, provided with efficient stirring and a distillation apparatus. The excess n-pentanol was then eliminated by distillation at a temperature of 110° C. under a partial vacuum of between approximately 125 mbar and 50 mbar. The distilled reaction medium was immediately diluted by adding a quantity of water in a manner such as to obtain a concentration of reaction medium of approximately 70%. After homogenization for 60 minutes at a temperature of 40° C., the composition (X₅) obtained was evacuated.

TABLE 2

| | Composition (X₅) |
|---|---|
| Appearance at 20° C. (Visual determination) | Clear orange liquid |
| Acid number (NFT standard 60204) | 0.7 |
| Dry extract hydroxyl value (USP XXI NF XVI | 840 |
| standard Jan. 1, 1995) | |
| pH at 5% (NFT standard 73206) | 5.5 |
| Water (% by weight) (NFT standard 73201) | 28.1% |
| Residual n-pentanol content (gas phase | 0.2% |
| chromatography), as a % by weight | |

2.2 Preparation of a Composition (X₆) Comprising a Composition of n-Pentyl Polyglucosides and a Composition of Caprylyl/Caprylic Polyglucoside (50/50)

100 grams of composition (X₅) obtained using the process described in section 2.2 of the present application and 118.8 grams of a composition of caprylyl/caprylic glucoside (50/50) [composition (X₃)], marketed with the trade name ORAMIX™ CG110 and having a dry extract of 60.5% were introduced into a jacketed glass reactor with a capacity of two litres in which a heat transfer fluid was circulated, provided with efficient stirring, at a temperature of 25° C.

The mixture was stirred for 30 minutes at 25° C., then the composition (X₆) obtained was evacuated out. The composition (X₆) obtained thereby comprised, with respect to 100% of its dry weight, 50% by weight of n-pentyl polyglucosides and 50% by weight of caprylyl/caprylic polyglucoside (50/50).

3) Evaluation of the Solubilizing Properties of n-Heptyl Polyglucosides and Comparative Compositions in Solubilizing Essential Oils and Perfumes in Water.

The solubilizing properties of composition (X₁) obtained using the process described in the preceding paragraph were evaluated in respect of essential oils and perfumes in water and compared with those of the composition of (2-ethylhexyl) polyglucosides, marketed with the trade name AG™ 6202 [composition (X₂)], with those of a composition of caprylyl/caprylic polyglucoside (50/50) [composition (X₃)], marketed with the trade name ORAMIX™ CG110, with those of a composition of hydrogenated and ethoxylated castor oil containing 40 moles of ethylene oxide [composition (X₄)], the INCI name of which is “PEG-40 hydrogenated castor oil” and the CAS number of which is 61788-85-0, marketed under the trade name SIMULSOL™1293, with that of composition (X₅) and with that of composition (X₆) using the evaluation method described below.

### 3.1—Experimental Protocol

For each hydrophobic “perfuming substance” (essential oil or perfume) to be solubilized, one gram dry matter of said hydrophobic perfuming substance and a quantity Yᵢ grams of surfactant composition to be evaluated as the solubilizing agent were introduced into a 150 cm³ glass flask. A quantity of distilled water was added at a temperature of 20° C. by way of complement in order to obtain a total mass of a hundred grams, so that the final mixture comprised 1% of hydrophobic perfuming substance to be solubilized and Yᵢ % by weight of solubilizing agent. The temperature was adjusted to 20° C. A magnetic bar stirrer was introduced into the glass flask and magnetic stirring was commenced at a rate of 80 revolutions per minute for a period of 60 minutes at a temperature (T₁) of 20° C. At the end of this period, the visual appearance obtained was recorded. In this manner, for each hydrophobic “perfuming substance”, the minimum quantity by weight required to solubilize 1% by weight of said hydrophobic “perfuming substance” was determined; in addition, for each minimum quantity of solubilizing agent, the turbidity of the solution obtained was measured at a temperature of 20° C. with the aid of a model DRT100B optical turbidimeter from HF Scientific, which had already been calibrated with a solution of Formazine (0.9 NTU). This experimental protocol was carried out for each of the compositions (X₁) in accordance with the invention and (X₂), (X₃), (X₄), (X₅) and (X₆) of the prior art.

### 3.2—Expression of Results

The visual appearances of each of the solutions obtained using the protocol of section 1.2.1 of the present application were recorded by the investigator and qualified as clear (C), cloudy (T) or cloudy-heterogeneous (T-H), depending on the case.

The turbidity measurements were expressed in NTU units.

3.3—Characterization of Solubilization, in an Aqueous Medium at a Temperature of 20° C., of Hydrophobic Perfuming Substances by the Composition (X₁) in Accordance with the Invention Compared with the Compositions (X₂), (X₃), (X₄), (X₅) and (X₆) of the Prior Art.

The appearances of the solutions prepared for each of the compositions (X₁) in accordance with the invention and (X₂), (X₃), (X₄), (X₅) and (X₆) of the prior art used to solubilize the perfuming substances at 20° C. were recorded by the investigator and noted in the tables below for the clove, palmarosa, cabreuva essential oils and for a perfume of the “floral note” type referred to as “Just delicious” from the Technicolor company.

TABLE 3.1
Appearance of solutions comprising 1% by weight of clove essential
oil and a variable quantity of (X₁), (X₂), (X₃), (X₄), (X₅) or (X₆)

| Fraction by weight | Appearance | | |
|---|---|---|---|
| of (X₁), (X₂) or (X₃) | of solution | | |
| (% dry matter) | (X₁) | (X₂) | (X₃) |
| 0% | T-H | T-H | T-H |
| 1.0% | T | T | T |
| 2.0% | C | T | T |
| 3.0% | C | T | T |
| 4.0% | n.m. | T | T |
| 5.0% | n.m. | T | T |
| 6.0% | n.m. | T | C |
| 7.0% | n.m. | T | C |
| 8.0% | n.m. | C | n.m. |
| 9.0% | n.m. | C | n.m. |
| Fraction by weight | Appearance | | |
| of (X₄), (X₅) or (X₆) | of solution | | |
| (% dry matter) | (X₄) | (X₅) | (X₆) |
| 0% | T-H | T-H | T-H |
| 1.0% | T | T | T |
| 2.0% | T | T | T |
| 3.0% | T | T | T |
| 4.0% | T | T | T |
| 5.0% | T | T | T |
| 6.0% | T | T | T |
| 7.0% | C | T | T |
| 8.0% | C | T | C |
| 9.0% | n.m. | n.m. | n.m. |

(*) n.m.: not measured

TABLE 3.2
Minimum weight ratio: clove essential oil/surfactant
composition Xi (Rₘᵢₙ) to obtain a clear solution.

| Composition | (X₁) | (X₂) | (X₃) | (X₄) | (X₅) | (X₆) |
|---|---|---|---|---|---|---|
| Rₘᵢₙᵢ | 1/2 | 1/8 | 1/6 | 1/7 | 1/8 | 1/8 |
| Turbidity | 3.6 | 4.8 | 27.3 | 18.5 | Insol- | 9.8 |
| (NTU) | | | | | uble | |

TABLE 4.1
Appearance of solution comprising 1% by weight of palmarosa
essential oil and a variable quantity of (X₁), (X₂), (X₃), (X₄), (X₅)
or (X₆)

| Fraction by weight | Appearance | | |
|---|---|---|---|
| of (X₁), (X₂) or (X₃) | of solution | | |
| (% dry matter) | (X₁) | (X₂) | (X₃) |
| 0% | T-H | T-H | T-H |
| 1.0% | T | T | T |
| 2.0% | T | T | T |
| 3.0% | T | T | T |
| 4.0% | T | T | T |
| 5.0% | T | T | T |
| 6.0% | C | T | T |
| 7.0% | C | T | T |
| 8.0% | n.m. | T | C |
| 9.0% | n.m. | C | C |
| Fraction by weight | Appearance | | |
| of (X₄), (X₅) or (X₆) | of solution | | |
| (% dry matter) | (X₄) | (X₅) | (X₆) |
| 0% | T-H | T-H | T-H |
| 1.0% | T | T | T |
| 2.0% | T | T | T |
| 3.0% | T | T | T |
| 4.0% | T | T | T |
| 5.0% | T | T | T |
| 6.0% | T | T | T |
| 7.0% | T | T | T |
| 8.0% | T | T | T |
| 9.0% | C | n.m. | n.m. |

(*) n.m.: not measured

TABLE 4.2
Minimum weight ratio: palmarosa essential oil/surfactant
composition Xi (Rₘᵢₙ), to obtain a clear solution.

| Composition | (X₁) | (X₂) | (X₃) | (X₄) | (X₅) | (X₆) |
|---|---|---|---|---|---|---|
| Rₘᵢₙᵢ | 1/6 | 1/9 | 1/8 | 1/9 | 1/8 | 1/8 |
| Turbidity | 11 | 5.2 | 25.5 | Insol- | Insol- | Insol- |
| (NTU) | | | | uble | uble | uble |

TABLE 5.1
Appearance of solutions comprising 1% by weight of cabreuva essential
oil and a variable quantity of (X₁), (X₂), (X₃), (X₄), (X₅) or (X₆)

| Fraction by weight | Appearance | | |
|---|---|---|---|
| of (X₄), (X₅) or (X₆) | of solution | | |
| (% dry matter) | (X₁) | (X₂) | (X₃) |
| 0% | T-H | T-H | T-H |
| 1.0% | T | T | T |
| 2.0% | T | T | T |
| 3.0% | T | T | T |
| 4.0% | T | T | C |
| 5.0% | T | T | C |
| 6.0% | C | T | n.m. |
| 7.0% | C | T | n.m. |
| 8.0% | n.m. | T | n.m. |
| 9.0% | n.m. | T | n.m. |
| Fraction by weight | Appearance | | |
| of (X₄), (X₅) or (X₆) | of solution | | |
| (% dry matter) | (X₄) | (X₅) | (X₆) |
| 0% | T-H | T-H | T-H |
| 1.0% | T | T | T |
| 2.0% | T | T | T |
| 3.0% | T | T | T |
| 4.0% | T | T | T |
| 5.0% | T | T | T |
| 6.0% | T | T | T |
| 7.0% | T | T | T |
| 8.0% | T | T | T |
| 9.0% | T | n.m. | n.m. |

(*) n.m.: not measured

TABLE 5.2
Minimum weight ratio: cabreuva essential oil/surfactant
composition Xi (Rₘᵢₙ) to obtain a clear solution.

| Composition | (X₁) | (X₂) | (X₃) | (X₄) | (X₅) | (X₆) |
|---|---|---|---|---|---|---|
| Rₘᵢₙᵢ | 1/6 | 1/9 | 1/3 | 1/9 | 1/8 | 1/8 |
| Turbidity | 3.8 | Insol- | 857 | Insol- | Insol- | Insol- |
| (NTU) | | uble | | uble | uble | uble |

Solubilizing clove essential oil requires a smaller quantity of composition (X₁) than those of compositions (X₂), (X₃), (X₄), (X₅) and (X₆).

The composition (X₁) comprising n-heptyl polyglucosides can be used to solubilize clove essential oil in water in an essential oil/composition (X₁) weight ratio of 1/2, while the comparative solubilizing compositions had to be employed in a larger quantity by weight, namely in an essential oil/composition (X₂) ratio of 1/8, in an essential oil/composition (X₃) ratio of 1/6, in an essential oil/composition (X₄) ratio of 1/7, in an essential oil/composition (X₆) ratio of 1/8; composition (X₅) could not solubilize clove essential oil in an essential oil/composition ratio (X₅) of less than or equal to 1/8.

The composition (X₁) comprising n-heptyl polyglucosides can be used to solubilize palmarosa essential oil in water in an essential oil/composition (X₁) weight ratio of 1/6, while the comparative solubilizing compositions had to be employed in a larger quantity by weight, namely in an essential oil/composition (X₂) ratio of 1/9, in an essential oil/composition (X₃) ratio of 1/8 and in an essential oil/composition (X₄) ratio of 1/9. The compositions (X₅) and (X₆) could not solubilize the palmarosa essential oil in water in an essential oil/composition (X₅) ratio and in an essential oil/composition (X₆) ratio of less than or equal to 1/8.

The composition (X₁) comprising n-heptyl polyglucosides can be used to solubilize cabreuva essential oil in an essential oil/composition (X₁) weight ratio of 1/6, while the comparative solubilizing compositions had to be employed in a larger quantity by weight, namely in an essential oil/composition (X₂) ratio greater than 1/9, in an essential oil/composition (X₃) ratio of 1/4 and in an essential oil/composition (X₄) ratio greater than 1/9; composition (X₃), however, suffered from the disadvantage of generating the formation of foam when stirring the solution comprising it, a phenomenon which was not observed with composition (X₁). The compositions (X₅) and (X₆) could not be used to solubilize cabreuva essential oil in water in an essential oil/composition (X₅) ratio and in an essential oil/composition (X₆) ratio of less than or equal to 1/8.

The composition (X₁) comprising n-heptyl polyglucosides could also be used to obtain an aqueous solution comprising 1% of clove essential oil and an aqueous solution of cabreuva essential oil that was more transparent, namely 3.6 NTU for the solution comprising clove essential oil and 3.8 NTU for the solution comprising cabreuva essential oil, than the comparative solubilizing compositions tested with these same essential oils.

1. A method of solubilizing a hydrophobic perfuming substance (A) or for a mixture of hydrophobic perfuming substances (A) in an aqueous solution, comprising mixing:
a) a composition represented by the formula (I):
R—O-(G)ₚ-H (I),
in which R represents then-heptyl radical, G represents the residue of a reducing sugar and p represents a decimal value greater than or equal to 1.05 and less than or equal to 5, said composition with formula (I) consisting of a mixture of compounds represented by the formulae (I₁), (I₂), (I₃), (I₄) and (I₅):
R—O-(G)₁-H (I₁)
R—O-(G)₂-H (I₂)
R—O-(G)₃-H (I₃)
R—O-(G)₄-H (I₄)
R—O-(G)₅-H (I₅)
in the respective molar proportions a₁, a₂, a₃, a₄ and a₅, such that each of the proportions a₁, a₂, a₃, a₄ and a₅ is greater than or equal to zero and less than or equal to one and such that the sum a₁+a₂+a₃+a₄+a₅ is equal to one,
b) water, and
c) a hydrophobic perfuming substance (A) or for a mixture of hydrophobic perfuming substances (A), said hydrophobic perfuming substance (A) being selected from the group constituted by essential oils that are rich in compounds carrying a phenolic function and essential oils that are rich in compounds carrying an alcohol function,
wherein the composition is a solubilizing agent in the aqueous solution, for the hydrophobic perfuming substance (A) or for the mixture of hydrophobic perfuming substances (A).

2. The method as claimed in claim 1, wherein said hydrophobic perfuming substance (A) is selected from the group consisting of benzyl alcohol, 3,7-dimethyl 1-octanol, isononyl alcohol, α-terpineol, menthol, linalol, citronellol, eucalyptol, geraniol, phytol, iso-phytol, tetrahydrolinalol, farnesol, carotol, nerol, globulol, vetiverol, nerolidol, dihydromyrcenol, tetrahydromyrcenol, fenchyl alcohol, benzyl dimethylcarbinol, cinnamic alcohol, 2-phenyl ethanol, undecavertol, anethole, safrole, isosafrole, eugenol, iso-eugenol, guaiacol, chavicol, estragole, cumic alcohol, thymol and p-cresol.

3. The method as claimed in claim 1, wherein, in formula (I), p represents a decimal value greater than or equal to 1.05 and less than or equal to 2.5.

4. The see method as claimed in claim 1, wherein, in formula (I), G represents the residue of a reducing sugar selected from the residues glucose, xylose and arabinose.

5. The see method as claimed in claim 1, wherein the ratio by weight between said hydrophobic perfuming substance (A) and the composition represented by the formula (I) is greater than or equal to 1/7 and less than or equal to 2/1.

6. An aqueous solution (C₂) comprising, with respect to 100% of its mass:
a)—0.05% to 70% by weight of at least one composition represented by the formula (I):
R—O-(G)ₚ-H (I)
in which R represents the n-heptyl radical, G represents the residue of a reducing sugar and p represents a decimal value greater than or equal to 1.05 and less than or equal to 5, said composition with formula (I) consisting of a mixture of compounds represented by the formulae (I₁), (I₂), (I₃), (I₄) and (I₅):
R—O-(G)₁-H (I₁)
R—O-(G)₂-H (I₂)
R—O-(G)₃-H (I₃)
R—O-(G)₄-H (I₄)
R—O-(G)₅-H (I₅)
in the respective molar proportions a₁, a₂, a₃, a₄ and a₅, such that the sum a₁+a₂+a₃+a₄+a₅ is equal to one and such that each of the proportions a₁, a₂, a₃, a₄ and a₅ is greater than or equal to zero and less than or equal to one;
b)—0.1% to 10% by weight of at least one hydrophobic perfuming substance (A) selected from the group consisting of essential oils that are rich in compounds carrying a phenolic function, essential oils that are rich in compounds carrying an alcohol function, benzyl alcohol, 3,7-dimethyl 1-octanol, isononyl alcohol, alpha terpineol, menthol, linalol, citronellol, eucalyptol, geraniol, phytol, iso-phytol, tetrahydrolinalol, farnesol, carotol, nerol, globulol, vetiverol, nerolidol, dihydromyrcenol, tetrahydromyrcenol, fenchyl alcohol, benzyl dimethylcarbinol, cinnamic alcohol, 2-phenyl ethanol, undecavertol, anethole, safrole, isosafrole, eugenol, iso-eugenol, guaiacol, chavicol, estragole, cumic alcohol, thymol, p-cresol, and mixtures thereof;
c)—20% to 99.85% by weight of water.

7. The aqueous solution (C₂) as claimed in claim 6, wherein the ratio by weight between said hydrophobic perfuming substance (A) and the composition represented by the formula (I) is greater than or equal to 1/7 and less than or equal to 2/1.

8. The aqueous solution (C₂) as claimed in claim 6, wherein the aqueous solution (C₂) has a turbidity greater than or equal to 1 NTU and less than or equal to 100 NTU, measured quantitatively by means of a nephelometric optical method.

9. A method for perfuming the skin, the hair, the scalp, the mucous membranes or clothing, comprising a step of applying to said skin, said hair, said scalp, said mucous membranes or said clothing of the aqueous solution (C₂) as claimed in claim 6.

10. A cosmetic composition (C₃) for topical use, comprising the aqueous solution (C₂) as claimed in claim 6, as a perfuming agent.
